(19) United States
(12) Patent Application Publication
(10) Pub. No.: US 2011/0301245 A1
(43) Pub. Date: Dec. 8, 2011

(54) (R)-2-(3,4-DIMETHOXYPHENOL)-2-ISOPROPYL-6-AZAHEPTANITRIL FORMULAITONS, ANDTHE USE OF SUCH FORMULATIONS IN THE TREATMENT OF CONDITIONS MEDIATED BY THE SEROTONIN TRANSPORTER

(75) Inventor: John DEVANE, Rosscommon (IE)

(73) Assignee: AGI THERAPEUTICS, PLC, Dublin (IE)

(21) Appl. No.: 12/850,329

(22) Filed: Aug. 4, 2010

Related U.S. Application Data
(63) Continuation-in-part of application No. 12/794,376, filed on Jun. 4, 2010, now abandoned.

(51) Int. Cl. A61K 31/277; A61P 9/00; A61P 9/12

(52) U.S. Cl. 514/654

(57) ABSTRACT

Methods comprising administering compositions comprising therapeutically effective amounts of the (R)-isomer of 2-(3,4-dimethoxyphenol)-2-isopropyl-6-azaheptanitril, a derivative thereof, or a pharmaceutically acceptable salt thereof, wherein the composition treats or prevents at least one condition having serotonin transporter activity in a subject in need thereof, and releases the (R)-isomer of 2-(3,4-dimethoxyphenol)-2-isopropyl-6-azaheptanitril, a derivative thereof, or a pharmaceutically acceptable salt thereof to exhibit an activity on the serotonin transporter.

### PRIOR RELATED APPLICATION

This application is a continuation-in-part of U.S. Ser. No. 12/794,376, filed Jun. 4, 2010, the entire disclosure of which is considered as being part of this application and is expressly incorporated by reference herein in its entirety.

### FIELD OF THE INVENTION

The present invention is generally directed to methods of administering compositions comprising therapeutically effective amounts of the (R)-isomer of 2-(3,4-dimethoxyphenol)-2-isopropyl-6-azaheptanitril, a derivative thereof, or a pharmaceutically acceptable salt thereof, wherein the composition treats or prevents at least one condition having serotonin transporter activity in a subject in need thereof, and releases the (R)-isomer of 2-(3,4-dimethoxyphenol)-2-isopropyl-6-azaheptanitril, a derivative thereof, or a pharmaceutically acceptable salt thereof to exhibit an activity on the serotonin transporter.

### BACKGROUND OF THE INVENTION

Pulmonary arterial hypertension (PAH) is a debilitating disease which is often fatal. PAH is characterized by a progressive increase in pulmonary vascular resistance which makes the right side of the heart work harder than normal, leading to right ventricular failure.

An in vivo model used to assess the effects of various agents on pulmonary arterial pressure and right ventricular hypertension in subjects with PAH has been developed (Rey et al., Current Protocols in Pharmacology, 5.56.1-5.56.11, September 2009, which document is incorporated by reference herein in its entirety). In particular, monocrotaline (Mct) has been found to induce progressive pulmonary hypertension, right ventricular overload, and right ventricular hypertrophy in rats after a single subcutaneous injection. The exposure to Mct leads to endothelial dysfunction and damage, which is associated with the clinical pathogenesis of PAH in humans.

2-(3,4-dimethoxyphenol)-2-isopropyl-6-azaheptanitril is one of thirteen known metabolites of verapamil, a calcium channel blocking drug used to treat cardiovascular conditions. 2-(3,4-dimethoxyphenol)-2-isopropyl-6-azaheptanitril is a secondary amine that is the product of N-dealkylation of verapamil.

Often referred to as D-617, 2-(3,4-dimethoxyphenol)-2-isopropyl-6-azaheptanitril has been regarded as devoid of any relevant pharmacology. For instance compared with verapamil, it has only 2.6% of the cardiovascular potency, in contrast with nor-verapamil (also known as “D591”), the N-demethylated metabolite, which has 21.9% the potency of verapamil. For this reason, nor-verapamil (not D-617) is routinely measured in circulating plasma along with parent verapamil to assess bioequivalence for FDA approval purposes.

Verapamil itself is a racemic drug with a chiral center that is maintained in the majority of its metabolites. Thus, D-617 exists as both R- and S-isomers. There is an established differential pharmacology between the isomers of verapamil, with the S-isomer known to be dominantly cardiovascularly potent. In contrast, the R-isomer has been shown to be intestinally selective.

Comprehensive assessment of the pharmacology of D-617 or its metabolites has been quite limited. For example, there have been no studies of any therapeutic activity of D-617 when administered in humans. The non-clinical studies conducted on D-617 to date have focused on its pharmacokinetic properties, particularly establishing that it is a substrate for, but not an inhibitor of, p-glycoprotein transport; that it is a substrate for but not a potent inhibitor of CYP-mediated metabolism; and that it does not block HERG-induced currents.

After administration of verapamil, D-617 is detected in plasma at levels similar or even exceeding those of verapamil and it is the highest detected metabolite in the urine.

### SUMMARY OF THE INVENTION

The present inventor has surprisingly discovered that (R)-2-(3,4-dimethoxyphenol)-2-isopropyl-6-azaheptanitril has activity on the serotonin transporter. When tested at a single high concentration (1×10⁻⁵M), (R)-2-(3,4-dimethoxyphenol)-2-isopropyl-6-azaheptanitril inhibited the serotonin transporter at a level of 100%. In contrast, little or no inhibition (universally <75%) was observed for the majority of the other receptors tested, including the traditional receptor targets of verapamil, e.g., calcium channels (data not included).

While verapamil has been reported to inhibit the serotonin transporter, this has typically been at concentrations similar to or higher than those used therapeutically to treat cardiovascular conditions (“Effect of calcium channel blockers on serotonin uptake,” Proc. Soc. Expt. Biol. Med. 1990: 193 (4), pp 326-330. Hill N S et al.) and therefore cannot be used as a selective serotonin transporter inhibitor. In addition R-verapamil has been shown to have co-potent activity on calcium channels, melatonin (MT1), and 5-HT_{2b} receptors and a lower potency of binding to the serotonin transporter, again preventing any selective serotonin transporter activity.

When detailed IC50 values were determined for inhibition of binding at the serotonin transporter, R-D617 (0.47×10⁻⁷M) was at least ten times more potent than S-verapamil (5.9×10⁻⁷M) and thirty times more potent than R-verapamil (16×10⁻⁷M). These findings were highly unexpected, and provide for the basis of a number of therapeutic indications. The present invention is based on this discovery, as well as others.

The invention provides methods of administering a composition comprising a therapeutically effective amount of the (R)-isomer of 2-(3,4-dimethoxyphenol)-2-isopropyl-6-azaheptanitril, a derivative thereof, or a pharmaceutically acceptable salt thereof, wherein the composition treats at least one condition having serotonin transporter activity in a subject in need thereof and releases the (R)-isomer of 2-(3,4-dimethoxyphenol)-2-isopropyl-6-azaheptanitril, a derivative thereof, or a pharmaceutically acceptable salt thereof to exhibit an activity on the serotonin transporter.

The invention further provides methods comprising administering a composition comprising a therapeutically effective amount of the (R)-isomer of 2-(3,4-dimethoxyphenol)-2-isopropyl-6-azaheptanitril, a derivative thereof, or a pharmaceutically acceptable salt thereof, wherein the composition prevents at least one condition having serotonin transporter activity in a subject in need thereof and releases the (R)-isomer of 2-(3,4-dimethoxyphenol)-2-isopropyl-6-azaheptanitril, a derivative thereof, or a pharmaceutically acceptable salt thereof to exhibit an activity on the serotonin transporter.

In the methods of the invention, the (R)-isomer of 2-(3,4-dimethoxyphenol)-2-isopropyl-6-azaheptanitril, a derivative thereof, or a pharmaceutically acceptable salt thereof, may be present in the composition in an amount ranging from about 1 mg to about 1000 mg, or from about 1 mg to 800 mg per day, or from about 1 mg to about 250 mg per day.

In the methods of the invention, the unbound plasma concentration range provided by the (R)-isomer of 2-(3,4-dimethoxyphenol)-2-isopropyl-6-azaheptanitril, a derivative thereof, or a pharmaceutically acceptable salt thereof, will generally range from about 1×10⁻⁵M to about 1×10⁻¹⁰M.

Conditions preventable and/or treatable by the present invention include, but are not limited to, a) diarrhea symptoms associated with carcinoid syndrome, chemotherapy, radiation, celiac disease, inflammatory bowel disease, mastocytosis, food allergies, and IBS; b) mucositis; c) nausea/vomiting including cyclic vomiting d) intestinal pain/distention/bloating; e) loss of appetite; f) facial flushing; g) headaches, including migraines and cluster headaches h) symptoms of pre-menstrual syndrome (“PMS”); i) cachexia; j) pulmonary arterial hypertension (also known as PAH), k) portal hypertension, l) depression and other CNS affective disorders including panic attacks, anxiety and social phobia, m) other psychiatric conditions such as mania or manic phases of psychotic conditions such as manic depression and diseases and/or conditions thereof of any of the foregoing.

Conditions preventable and/or treatable by the present invention also include conditions associated with increased arterial pressure and/or cardiac hypertrophy. For example, conditions preventable and/or treatable by the present invention include increased pulmonary vascular resistance and/or ventricular failure. Other examples of conditions preventable and/or treatable by the present invention include increased pulmonary arterial pressure and right ventricular hypertrophy.

Conditions preventable and/or treatable by the present invention also include conditions associated with: a) thrombus formation; b) platelet aggregation, and diseases and/or conditions thereof of any of the foregoing. In some embodiments, administering the composition of the invention reduces thrombus formation by reducing platelet aggregation. The compositions of the invention may be administered by, for example, oral, nasal, rectal, intravaginal, parenteral, buccal, sublingual, or topical administration. The inventive compositions may take the form of a tablet, a capsule, a suppository, and an enema.

The compositions of the invention may further include excipient(s). Excipients include, but are not limited to, starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

The compositions of the invention may further include additional pharmaceutically active agents, other than the (R)-isomer of 2-(3,4-dimethoxyphenol)-2-isopropyl-6-azaheptanitril, a derivative thereof, or a pharmaceutically acceptable salt thereof. Compositions of the invention include, but are not limited to, immediate release, modified release, sustained release, controlled release, and any combination thereof. Compositions may be administered one to five times per day.

A composition comprising from about 1 mg to about 1000 mg of the (R)-isomer of 2-(3,4-dimethoxyphenol)-2-isopropyl-6-azaheptanitril and at least one pharmaceutically acceptable excipient.

These and other objects, aspects, embodiments and features of the invention will become more fully apparent when read in conjunction with the following detailed description.

### BRIEF DESCRIPTION OF THE DRAWINGS

FIG. 1 shows a competition curve obtained for (R)-2-(3,4-dimethoxyphenol)-2-isopropyl-6-azaheptanitril on the human serotonin transporter.

FIG. 2 shows the effects of the (R)-isomer of 2-(3,4-dimethoxyphenol)-2-isopropyl-6-azaheptanitril on serotonin uptake.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Throughout this disclosure, reference will be made to compounds according to the invention. Reference to such compounds, in the specification and claims, includes esters and salts of such compounds. Thus, even if not explicitly recited, such esters and salts are contemplated, and encompassed, by reference to the compounds themselves.

All percent measurements in this application, unless otherwise stated, are measured by weight based upon 100% of a given sample weight. Thus, for example, 30% represents 30 weight parts out of every 100 weight parts of the sample.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

Serotonin may also be referred to herein as 5-hydroxtryptamine, or 5-HT.

Reference to the “(R)-isomer of 2-(3,4-dimethoxyphenol)-2-isopropyl-6-azaheptanitril,” or “(R)-2-(3,4-dimethoxyphenol)-2-isopropyl-6-azaheptanitril,” means a composition enriched in the (R)-stereoisomer of 2-(3,4-dimethoxyphenol)-2-isopropyl-6-azaheptanitril as compared to the amount of the (S)-stereoisomer present. For example, the composition may contain greater than 50%, 55%, or at least about 60% of the (R)-2-(3,4-dimethoxyphenol)-2-isopropyl-6-azaheptanitril stereoisomer by weight, based on the total weight of 2-(3,4-dimethoxyphenol)-2-isopropyl-6-azaheptanitril. In some embodiments, the amount of (R)-2-(3,4-dimethoxyphenol)-2-isopropyl-6-azaheptanitril may be, for example, at least about 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100%, or any fraction thereof (i.e., 90.1%, 90.2%, etc.), of (R)-2-(3,4-dimethoxyphenol)-2-isopropyl-6-azaheptanitril by weight, based on the total weight of 2-(3,4-dimethoxyphenol)-2-isopropyl-6-azaheptanitril. In a particular embodiment, the amount of (R)-2-(3,4-dimethoxyphenol)-2-isopropyl-6-azaheptanitril may be greater than 99%, 99.1%, 99.2%, 99.3%, 99.4%, 99.5%, 99.6%, 99.7%, 99.8%, 99.9% or may be 100%, by weight, based on the total weight of 2-(3,4-dimethoxyphenol)-2-isopropyl-6-azaheptanitril. These terms also define the amount of any pharmaceutically acceptable salts of (R)-2-(3,4-dimethoxyphenol)-2-isopropyl-6-azaheptanitril. “(R)-2-(3,4-dimethoxyphenol)-2-isopropyl-6-azaheptanitril” and “(R)-isomer of 2-(3,4-dimethoxyphenol)-2-isopropyl-6-azaheptanitril” may be used interchangeably herein.

As used herein, the term “immediate release” describes a formulation that releases the drug upon dissolution, without significant delay. In most embodiments, such formulations would release drug in the upper GI, including the mouth, esophagus, and/or stomach.

As used herein, the phrase “modified-release” formulation or dosage form includes a pharmaceutical preparation that achieves a desired release of the drug from the formulation. For example, a modified-release formulation may extend the influence or effect of a therapeutically effective dose of a pharmaceutically active compound in a patient. Such formulations are referred to herein as “extended-release formulations.” In addition to maintaining therapeutic levels of the pharmaceutically active compound, a modified-release formulation may also be designed to delay the release of the active compound for a specified period. Such compounds are referred to herein as “delayed onset” of “delayed release” formulations or dosage forms. Still further, modified-release formulations may exhibit properties of both delayed and extended release formulations, and thus be referred to as “delayed-onset, extended-release” formulations.

As used herein, the term “pharmaceutically acceptable excipient” includes compounds that are compatible with the other ingredients in a pharmaceutical formulation and not injurious to the subject when administered in normal, or therapeutically effective, amounts.

As used herein, the term “pharmaceutically acceptable salt” includes salts that are physiologically tolerated by a subject. Such salts are typically prepared from an inorganic and/or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like.

The phrase “therapeutically effective amount of (R)-2-(3,4-dimethoxyphenol)-2-isopropyl-6-azaheptanitril,” as used herein, refers to the amount of (R)-2-(3,4-dimethoxyphenol)-2-isopropyl-6-azaheptanitril (or derivative thereof or a pharmaceutically acceptable salt thereof), which alone or in combination with other drugs, provides any therapeutic benefit in the prevention, treatment, and/or management of diseases and/or conditions associated with the activity of the serotonin transporter.

The term “antagonist,” as used herein, refers to agents or drugs that neutralize or impede the action or effects of others, e.g., a drug that binds to a receptor without eliciting a biological response and effectively blocking the binding of a substance that could elicit such a response. Antagonists may be competitive and reversible by reversibly binding to a region of a receptor in common with the agonist or competitive and irreversible by covalently binding to the agonist binding site. Antagonists may also be non-competitive where the antagonist binds to an allosteric site on the receptor or an associated ion channel.

The invention is directed to methods for treating, preventing, and/or managing diseases and/or conditions associated with the activity of the serotonin transporter, comprising administering a therapeutically effective amount of (R)-2-(3,4-dimethoxyphenol)-2-isopropyl-6-azaheptanitril, a derivative thereof, or a pharmaceutically acceptable salt thereof. It is noted that such treatments are achieved by administering to a patient in need of treatment (R)-2-(3,4-dimethoxyphenol)-2-isopropyl-6-azaheptanitril, a derivative thereof, or a pharmaceutically acceptable salt thereof. Thus, a plasma concentration of (R)-2-(3,4-dimethoxyphenol)-2-isopropyl-6-azaheptanitril is achieved in a patient without the co-occurrence of a plasma level of R-verapamil. Therefore, the present method achieves therapeutic levels of (R)-2-(3,4-dimethoxyphenol)-2-isopropyl-6-azaheptanitril, in the absence of therapeutic levels of R-verapamil.

Conditions treatable by the present invention include, but are not limited to, a) diarrhea symptoms associated with carcinoid syndrome, chemotherapy, radiation, celiac disease, inflammatory bowel disease, mastocytosis, food allergies, and IBS; b) mucositis; c) nausea/vomiting including cyclic vomiting d) intestinal pain/distention/bloating; e) loss of appetite; f) facial flushing; g) headaches, including migraines and cluster headaches h) symptoms of pre-menstrual syndrome (“PMS”); i) cachexia; j) pulmonary arterial hypertension (also known as PAH), k) portal hypertension, l) depression and other CNS affective disorders including panic attacks, anxiety and social phobia, m) other psychiatric conditions such as mania or manic phases of psychotic conditions such as manic depression and diseases and/or conditions thereof of any of the foregoing.

Conditions preventable and/or treatable by the present invention also include conditions associated with increased arterial pressure and/or cardiac hypertrophy. For example, conditions preventable and/or treatable by the present invention include increased pulmonary vascular resistance and/or ventricular failure. Other examples of conditions preventable and/or treatable by the present invention include increased pulmonary arterial pressure and right ventricular hypertrophy.

In at least one embodiment, (R)-2-(3,4-dimethoxyphenol)-2-isopropyl-6-azaheptanitril, a derivative thereof, or a pharmaceutically acceptable salt thereof, is provided in a composition for use in treating, preventing, and/or managing diseases and/or conditions associated with the activity of the serotonin transporter. Such compositions will generally also include at least one pharmaceutically acceptable excipient. Suitable excipients are known to those of skill in the art and examples are described, for example, in the Handbook of Pharmaceutical Excipients (Kibbe (ed.), 3rd Edition (2000), American Pharmaceutical Association, Washington, D.C.), and Remington's Pharmaceutical Sciences (Gennaro (ed.), 20th edition (2000), Mack Publishing, Inc., Easton, Pa.), which, for their disclosures relating to excipients and dosage forms, are incorporated herein by reference. For example, suitable excipients include, but are not limited to, starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, plasticizers, gelling agents, thickeners, hardeners, setting agents, suspending agents, surfactants, humectants, carriers, stabilizers, antioxidants, and combinations thereof.

The pharmaceutical compositions of the invention are typically provided in dosage forms that are suitable for administration to a subject by a desired route. A number of suitable dosage forms are described below, but are not meant to include all possible choices. One of skill in the art is familiar with the various dosage forms that are suitable for use in the present invention, as described, for example, in Remington's Pharmaceutical Sciences, which has been incorporated by reference above. The most suitable route in any given case will depend on the nature and severity of the disease and/or condition being prevented, treated, and/or managed. For example, the pharmaceutical compositions may be formulated for administration orally, nasally, rectally, intravaginally, parenterally, intracistemally, and topically, including buccally and sublingually.

Formulations suitable for oral administration include, but are not limited to, capsules, cachets, pills, tablets, lozenges (using a flavored basis, usually sucrose and acacia or tragacanth), powders, granules, solutions, suspensions in an aqueous or non-aqueous liquid, oil-in-water or water-in-oil liquid emulsions, elixirs, syrups, pastilles (using an inert base, such as gelatin and glycerin, or sucrose and acacia), mouth washes, pastes, and the like; each containing a predetermined amount of (R)-2-(3,4-dimethoxyphenol)-2-isopropyl-6-azaheptanitril, a derivative thereof, or a pharmaceutically acceptable salt thereof, to provide a therapeutic amount of the drug in one or more doses.

In solid dosage forms for oral administration (capsules, tablets, pills, powders, granules and the like), the (R)-2-(3,4-dimethoxyphenol)-2-isopropyl-6-azaheptanitril, derivative thereof, or pharmaceutically acceptable salt thereof is typically mixed with one or more pharmaceutically-acceptable excipients, including carriers, such as sodium citrate or dicalcium phosphate; fillers or extenders, such as starches, spray-dried or anhydrous lactose, sucrose, glucose, mannitol, dextrose, sorbitol, cellulose (e.g., microcrystalline cellulose; AVICEL™), dehydrated or anhydrous dibasic calcium phosphate, and/or silicic acid; binders, such as acacia, alginic acid, carboxymethylcellulose (sodium), cellulose (microcrystalline), dextrin, ethylcellulose, gelatin, glucose (liquid), guar gum, hydroxypropyl cellulose, hydroxypropyl methylcellulose, methylcellulose (e.g., methylcellulose 2910), polyethylene oxide, povidone, starch (pregelatinized) or syrup; humectants, such as glycerol; disintegrating agents, such as agar, calcium carbonate, potato or tapioca starch, alginic acid, certain silicates, pregelatinized starch, sodium starch glycolate (EXPLOTAB™), crosslinked providone, crosslinked sodium carboxymethylcellulose, clays, microcrystalline cellulose (e.g., AVICEL™), alginates, gums, and/or sodium carbonate; solution retarding agents, such as paraffin; absorption accelerators, such as quaternary ammonium compounds; wetting agents, such as cetyl alcohol or glycerol monostearate; absorbents, such as kaolin and bentonite clay; lubricants, such as talc, calcium stearate, magnesium stearate, solid polyethylene glycols, steric acid, sodium stearyl fumarate, magnesium lauryl sulfate, hydrogenated vegetable oil, and/or sodium lauryl sulfate; glidants, such as calcium silicate, magnesium silicate, colloidal anhydrous silica, and/or talc; flavoring agents, such as synthetic flavor oils and flavoring aromatics, natural oils, extracts from plant leaves, flowers, and fruits, including cinnamon oil, oil of wintergreen, peppermint oils, bay oil, anise oil, eucalyptus, thyme oil, vanilla, citrus oil (e.g., lemon, orange, grape, lime, and grapefruit), fruit essences (e.g., apple, banana, pear, peach, strawberry, raspberry, cherry, plum, pineapple, apricot, as so forth); coloring agents and/or pigments, such as titanium dioxide and/or dyes approved for use in food and pharmaceuticals; buffering agents; dispersing agents; preservatives; and/or diluents. The aforementioned excipients are given as examples only and are not meant to include all possible choices.

Any of these solid dosage forms may optionally be scored or prepared with coatings and shells, such as enteric coatings, and coatings for modifying the rate of release, examples of which are well known in the pharmaceutical-formulating art. For example, such coatings may comprise sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, wax, or zein. In one embodiment, the coating material comprises hydroxypropyl methylcellulose. The coating material may further comprise anti-adhesives, such as talc; plasticizers (depending on the type of coating material selected), such as castor oil, diacetylated monoglycerides, dibutyl sebacate, diethyl phthalate, glycerin, polyethylene glycol, propylene glycol, triacetin, triethyl citrate; opacifiers, such as titanium dioxide; and/or coloring agents and/or pigments. The coating process may be carried out by any suitable means, for example, by using a perforated pan system such as the GLATT™, ACCELACOTA™, and/or HICOATER™ apparatuses.

Tablets may be formed by any suitable process, examples of which are known to those of ordinary skill in the art. For example, the ingredients may be dry-granulated or wet-granulated by mixing in a suitable apparatus before tabletting. Granules of the ingredients to be tabletted may also be prepared using suitable spray/fluidization or extrusion/spheronization techniques.

With quick-release tablets, the choice of excipients generally allows a fast dissolution. The tablets may be conventional instant release tablets designed to be taken whole in the typical administration manner (i.e., with sufficient amount of water to facilitate swallowing). Alternatively, the tablets may be formulated with suitable excipients to act as a fast dissolving and/or fast melting tablet in the oral cavity. Also, the tablet can be in the form of a chewable or effervescent dosage form. With effervescent dosage forms, the tablet is typically added to a suitable liquid that causes it to disintegrate, dissolve, and/or disperse.

Tablets typically are designed to have an appropriate hardness and friability to facilitate manufacture on an industrial scale using equipment to produce tablets at high speed. Also, the tablets can be packed or filled in all kinds of containers. If the tablet has an insufficient hardness or is friable, the tablet that is taken by the subject may be broken or crumbled into powder. As a consequence of this insufficient hardness or friability, the subject can no longer be certain that the amount of the dose is correct. It should be noted that the hardness of tablets, amongst other properties, can be influenced by the shape of the tablets. Different shapes of tablets may be used according to the present invention. Tablets may be circular, oblate, oblong, or any other shape that is known in the art. The shape of the tablets may also influence the disintegration rate.

Any of the solid compositions may be encapsulated in soft or hard gelatin capsules using any of the excipients described here. For example, the encapsulated dosage form may include fillers, such as lactose and microcrystalline; glidants, such as colloidal silicon dioxide and talc; lubricants, such as magnesium stearate; and disintegrating agents, such as starch (e.g., maize starch). Using capsule filling equipment, the ingredients to be encapsulated can be milled together, sieved, mixed, packed together, and then delivered into a capsule. The lubricants may be present in any amount, such as from about 0.5% (w/w) to about 2.0% (w/w). In one embodiment, the lubricant is about 1.25% (w/w) of the content of the capsule.

(R)-2-(3,4-dimethoxyphenol)-2-isopropyl-6-azaheptanitril, a derivative thereof, or a pharmaceutically acceptable salt thereof may also be formulated into a liquid dosage form for oral administration. Suitable formulations include emulsions, microemulsions, solutions, suspensions, syrups, and elixirs. These formulations optionally include diluents commonly used in the art, such as, for example, water and/or other solvents, solubilizing agents and emulsifiers, including, but not limited to, ethyl alcohol, isopropyl alcohol, ethyl carbonate, ethyl acetate, benzyl alcohol, benzyl benzoate, propylene glycol, 1,3-butylene glycol, oils, glycerol, tetrahydrofuryl alcohol, polyethylene glycols, fatty acid esters of sorbitan, and mixtures thereof. In addition, the liquid formulations optionally include adjuvants such as wetting agents, emulsifying and suspending agents, sweetening, flavoring, coloring, perfuming, and preservative agents. Suitable suspension agents include, but are not limited to, ethoxylated isostearyl alcohols, polyoxyethylene sorbitol and sorbitan esters, microcrystalline cellulose, aluminum metahydroxide, bentonite, agar-agar and tragacanth, xanthan gum, hydroxypropylmethylcellulose, methylcellulose, carrageenan, sodium carboxymethyl cellulose, and sodium carboxymethyl cellulose/microcrystalline cellulose mixtures, sodium carboxymethyl cellulose/microcrystalline cellulose mixtures, and/or mixtures thereof. In one embodiment, the suspending agent comprises xanthan gum, carrageenan, sodium carboxymethyl cellulose/microcrystalline cellulose mixtures, and/or mixtures thereof. In another embodiment, the suspending agent is AVICEL™ RC591, AVICEL™ RC581, and/or AVICEL™ CL611 (Avicel is a trademark of FMC Corporation); and/or RC591, RC581 and CL611 (mixtures of microcrystalline cellulose and sodium carboxymethyl cellulose).

The amount of suspending agent present will generally vary according to the particular suspending agent used and the presence or absence of other ingredients, which have an ability to act as a suspending agent or contribute significantly to the viscosity of the composition. The suspension may also contain ingredients to improve its taste, for example sweeteners; bitter-taste maskers, such as sodium chloride; taste-masking flavors, such as contramarum; flavor enhancers, such as monosodium glutamate; and flavoring agents. Examples of sweeteners include bulk sweeteners, such as sucrose, hydrogenated glucose syrup, the sugar alcohols sorbitol and xylitol; and sweetening agents such as sodium cyclamate, sodium saccharin, aspartame, and ammonium glycyrrhizinate. The liquid formulations may further comprise at least one buffering agent, as needed, to maintain the desired pH. The liquid formulations of the present invention may also be filled into soft gelatin capsules. For example, the liquid may include a solution, suspension, emulsion, microemulsion, precipitate, or any other desired liquid media carrying (R)-2-(3,4-dimethoxyphenol)-2-isopropyl-6-azaheptanitril, derivative thereof, or pharmaceutically acceptable salt thereof. The liquid may be designed to improve the solubility of the (R)-2-(3,4-dimethoxyphenol)-2-isopropyl-6-azaheptanitril, derivative thereof, or pharmaceutically acceptable salt thereof upon release, or may be designed to form a drug-containing emulsion or dispersed phase upon release. Examples of such techniques are well known in the art. Soft gelatin capsules may be coated, as desired, with a functional coating, as described herein, to delay the release of the drug.

For rectal or vaginal administration, the composition may be provided as a suppository. Suppositories optionally comprise at least one non-irritating excipient, for example, polyethylene glycol, a suppository wax, or a salicylate. Such excipients may be selected on the basis of desirable physical properties. For example, a compound that is solid at room temperature but liquid at body temperature will melt in the rectum or vaginal cavity and release the active compound. The formulation may alternatively be provided as an enema for rectal delivery. Formulations suitable for vaginal administration also include pessaries, tampons, creams, gels, pastes, foams, or spray formulations containing such carriers, examples of which are known in the art.

Formulations suitable for topical or transdermal administration include powders, sprays, ointments, pastes, creams, lotions, gels, solutions, patches, and inhalants. Such formulations optionally contain excipients such as animal and vegetable fats, oils, waxes, paraffins, starch, tragacanth, cellulose derivatives, polyethylene glycols, silicones, bentonites, silicic acid, talc, zinc oxide, or mixtures thereof. Powders and sprays may also contain excipients such as lactose, talc, silicic acid, aluminum hydroxide, calcium silicates and polyamide powder. Additionally, sprays may contain propellants, such as chlorofluoro-hydrocarbons and volatile unsubstituted hydrocarbons, such as butane and propane.

Transdermal patches have the added advantage of providing controlled delivery of the mixture of the invention to the body. Such dosage forms can be made by dissolving, dispersing or otherwise incorporating a pharmaceutical composition containing 2-(3,4-dimethoxyphenol)-2-isopropyl-6-azaheptanitril, a derivative thereof or a pharmaceutically acceptable salt thereof in a suitable medium, such as an elastomeric matrix material. Absorption enhancers can also be used to increase the flux of the mixture across the skin. The rate of such flux can be controlled by either providing a rate-controlling membrane or dispersing the compound in a polymer matrix or gel.

For parenteral administration, such as administration by injection (including, but not limited to, subcutaneous, bolus injection, intramuscular, intraperitoneal, and intravenous), the pharmaceutical compositions may be formulated as isotonic suspensions, solutions or emulsions in oily or aqueous vehicles, and may contain formulatory agents such as suspending, stabilizing, or dispersing agents. Alternatively, the compositions may be provided in dry form such as a powder, crystalline or freeze-dried solid for reconstitution with sterile pyrogen-free water or isotonic saline before use. They may be presented, for example, in sterile ampoules or vials.

Examples of suitable aqueous and nonaqueous excipients include water, ethanol, polyols (such as glycerol, propylene glycol, polyethylene glycol, and the like), methacrylate), poly(ethyl methacrylate), poly(butyl methacrylate), poly(isobutyl methacrylate), and poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(ethylene oxide), poly(ethylene terephthalate), poly(vinyl isobutyl ether), poly(vinyl acetate), poly(vinyl chloride) or polyurethane, and/or mixtures thereof.

Suitable pharmaceutically acceptable excipients include, but are not limited to, carriers, such as sodium citrate and dicalcium phosphate; fillers or extenders, such as stearates, silicas, gypsum, starches, lactose, sucrose, glucose, mannitol, talc, and silicic acid; binders, such as hydroxypropyl methylcellulose, hydroxymethyl cellulose, alginates, gelatin, polyvinyl pyrrolidone, sucrose and acacia; humectants, such as glycerol; disintegrating agents, such as agar, calcium carbonate, potato and tapioca starch, alginic acid, certain silicates, EXPLOTAB™, crospovidone, and sodium carbonate; solution retarding agents, such as paraffin; absorption accelerators, such as quaternary ammonium compounds; wetting agents, such as cetyl alcohol and glycerol monostearate; absorbents, such as kaolin and bentonite clay; lubricants, such as talc, calcium stearate, magnesium stearate, solid polyethylene glycols, and sodium lauryl sulfate; stabilizers, such as fumaric acid; coloring agents; buffering agents; dispersing agents; preservatives; organic acids; and organic bases. The aforementioned excipients are given as examples only and are not meant to include all possible choices. Additionally, many excipients may have more than one role or function, or be classified oils, injectable organic esters, and mixtures thereof. Proper fluidity can be maintained, for example, by the use of coating materials and surfactants.

These compositions may also contain adjuvants such as preservatives, wetting agents, emulsifying agents, and dispersing agents. Prevention of the action of microorganisms may be achieved by the inclusion of various antibacterial and/or antifungal agents, for example, paraben, chlorobutanol, phenol sorbic acid, and the like. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monostearate and gelatin.

In order to prolong the therapeutic effect of a drug, it is often desirable to slow the absorption of the drug from a subcutaneous or intramuscular injection. This may be accomplished by the use of a liquid suspension of crystalline or amorphous material having low solubility. The rate of absorption of the drug then depends upon its rate of dissolution which, in turn, may depend upon crystal size and crystalline form. Alternatively, delayed absorption of a parenterally-administered form can be accomplished by dissolving or suspending the drug in an oil vehicle.

In addition to the common dosage forms described above, the compositions of the present invention may be formulated into a dosage form that modifies the release of 2-(3,4-dimethoxyphenol)-2-isopropyl-6-azaheptanitril, a derivative thereof or a pharmaceutically acceptable salt thereof. Examples of suitable modified release formulations, which may be used in accordance with the present invention include, but are not limited to, matrix systems, osmotic pumps, and membrane controlled dosage forms. These formulations typically comprise 2-(3,4-dimethoxyphenol)-2-isopropyl-6-azaheptanitril, a derivative thereof or a pharmaceutically acceptable salt thereof. Suitable pharmaceutically acceptable salts are discussed above.

Different types of modified dosage forms are briefly described below. A more detailed discussion of such forms may also be found in, for example The Handbook of Pharmaceutical Controlled Release Technology, D. L. Wise (ed.), Marcel Dekker, Inc., New York (2000); and also in Treatise on Controlled Drug Delivery: Fundamentals, Optimization, and Applications, A. Kydonieus (ed.), Marcel Dekker, Inc., New York, (1992), the relevant contents of each of which is hereby incorporated by reference for this purpose. Examples of modified release dosage forms are also described, for example, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, the disclosures of which, for their discussions of pharmaceutical formulations, are incorporated herein by reference.

Advantages of modified-release formulations may include extended activity of the drug, reduced dosage frequency, increased patient compliance, and the ability to deliver the drug to specific sites in the intestinal tract. Suitable components (e.g., polymers, excipients, etc.) for use in modified-release formulations, and methods of producing the same, are also described, e.g., in U.S. Pat. No. 4,863,742, which is incorporated by reference for these purposes.

Matrix-Based Dosage Forms

In some embodiments, the modified release formulations of the present invention are provided as matrix-based dosage forms. Matrix formulations according to the invention may include hydrophilic, e.g., water-soluble, and/or hydrophobic, e.g., water-insoluble, polymers. The matrix formulations of the present invention may optionally be prepared with functional coatings, which may be enteric, e.g., exhibiting a pH-dependent solubility, or non-enteric, e.g., exhibiting a pH-independent solubility.

Matrix formulations of the present invention may be prepared by using, for example, direct compression or wet granulation. A functional coating, as noted above, may then be applied in accordance with the invention. Additionally, a barrier or sealant coat may be applied over a matrix tablet core prior to application of a functional coating. The barrier or sealant coat may serve the purpose of separating an active ingredient from a functional coating, which may interact with the active ingredient, or it may prevent moisture from contacting the active ingredient. Details of barriers and sealants are provided below.

In a matrix-based dosage form in accordance with the present invention, 2-(3,4-dimethoxyphenol)-2-isopropyl-6-azaheptanitril and optional pharmaceutically acceptable excipient(s) are dispersed within a polymeric matrix, which typically comprises at least one water-soluble polymer and/or at least one water-insoluble polymer. The drug may be released from the dosage form by diffusion and/or erosion.

Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate cellulose propionate, cellulose acetate propionate, cellulose acetate butyrate, cellulose acetate phthalate, cellulose triacetate, poly(methyl in more than one group; the classifications are descriptive only, and not intended to limit any use of a particular excipient.

In one embodiment, a matrix-based dosage form comprises 2-(3,4-dimethoxyphenol)-2-isopropyl-6-azaheptanitril; a filler, such as starch, lactose, or microcrystalline cellulose (AVICEL™); a binder/controlled-release polymer, such as hydroxypropyl methylcellulose or polyvinyl pyrrolidone; a disintegrant, such as, EXPLOTAB™, crospovidone, or starch; a lubricant, such as magnesium stearate or stearic acid; a surfactant, such as sodium lauryl sulfate or polysorbates; and a glidant, such as colloidal silicon dioxide (AEROSIL™) or talc.

The amounts and types of polymers, and the ratio of water-soluble polymers to water-insoluble polymers in the inventive formulations are generally selected to achieve a desired release profile of 2-(3,4-dimethoxyphenol)-2-isopropyl-6-azaheptanitril, a derivative thereof or a pharmaceutically acceptable salt thereof. For example, by increasing the amount of water-insoluble-polymer relative to the amount of water-soluble polymer, the release of the drug may be delayed or slowed. This is due, in part, to an increased impermeability of the polymeric matrix, and, in some cases, to a decreased rate of erosion during transit through the GI tract.

Osmotic Pump Dosage Forms

In another embodiment, the modified release formulations of the present invention are provided as osmotic pump dosage forms. In an osmotic pump dosage form, a core containing 2-(3,4-dimethoxyphenol)-2-isopropyl-6-azaheptanitril, a derivative thereof or a pharmaceutically acceptable salt thereof, and optionally at least one osmotic excipient is typically encased by a selectively permeable membrane having at least one pore or orifice. The selectively permeable membrane is generally permeable to water, but impermeable to the drug. When the system is exposed to body fluids, water penetrates through the selectively permeable membrane into the core containing the drug and optional osmotic excipients. The osmotic pressure increases within the dosage form. Consequently, the drug is released through the pores or orifice(s) in an attempt to equalize the osmotic pressure across the selectively permeable membrane.

In more complex pumps, the dosage form may contain two internal compartments in the core. The first compartment contains the drug and the second compartment may contain a polymer, which swells on contact with aqueous fluid. After ingestion, this polymer swells into the drug-containing compartment, diminishing the volume occupied by the drug, thereby delivering the drug from the device at a controlled rate over an extended period of time. Such dosage forms are often used when a zero order release profile is desired.

Osmotic pumps are well known in the art. For example, U.S. Pat. Nos. 4,088,864, 4,200,098, and 5,573,776, each of which is hereby incorporated by reference for this purpose, describe osmotic pumps and methods of their manufacture. The osmotic pumps useful in accordance with the present invention may be formed by compressing a tablet of an osmotically active drug, or an osmotically inactive drug in combination with an osmotically active agent, and then coating the tablet with a selectively permeable membrane, which is permeable to an exterior aqueous-based fluid but impermeable to the drug and/or osmotic agent.

At least one delivery orifice may be drilled through the selectively permeable membrane wall. Alternatively, at least one orifice in the wall may be formed by incorporating leachable pore-forming materials in the wall. In operation, the exterior aqueous-based fluid is imbibed through the selectively permeable membrane wall and contacts the drug to form a solution or suspension of the drug. The drug solution or suspension is then pumped out through the orifice as fresh fluid is imbibed through the selectively permeable membrane.

Typical materials for the selectively permeable membrane include selectively permeable polymers known in the art to be useful in osmosis and reverse osmosis membranes, such as cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose dipalmitate, cellulose dioctanoate, cellulose dicaprylate, cellulose dipentanlate, cellulose acetate valerate, cellulose acetate succinate, cellulose propionate succinate, methyl cellulose, cellulose acetate p-toluene sulfonate, cellulose acetate butyrate, lightly cross-linked polystyrene derivatives, cross-linked poly(sodium styrene sulfonate), poly(vinylbenzyltrimethyl ammonium chloride), cellulose acetate, cellulose diacetate, cellulose triacetate, and/or mixtures thereof.

The osmotic agents that can be used in the pump are typically soluble in the fluid that enters the device following administration, resulting in an osmotic pressure gradient across the selectively permeable wall against the exterior fluid. Suitable osmotic agents include, but are not limited to, magnesium sulfate, calcium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glucose, hydrophilic polymers such as cellulose polymers, and/or mixtures thereof.

As discussed above, the osmotic pump dosage form may contain a second compartment containing a swellable polymer. Suitable swellable polymers typically interact with water and/or aqueous biological fluids, which causes them to swell or expand to an equilibrium state. Acceptable polymers exhibit the ability to swell in water and/or aqueous biological fluids, retaining a significant portion of such imbibed fluids within their polymeric structure, so as into increase the hydrostatic pressure within the dosage form. The polymers may swell or expand to a very high degree, usually exhibiting a 2- to 50-fold volume increase. The polymers can be non-cross-linked or cross-linked. In one embodiment, the swellable polymers are hydrophilic polymers. Suitable polymers include, but are not limited to, poly(hydroxy alkyl methacrylate) having a molecular weight of from 30,000 to 5,000,000; kappa-carrageenan; polyvinylpyrrolidone having a molecular weight of from 10,000 to 360,000; anionic and cationic hydrogels; polyelectrolyte complexes; poly(vinyl alcohol) having low amounts of acetate, cross-linked with glyoxal, formaldehyde, or glutaraldehyde, and having a degree of polymerization from 200 to 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; and/or mixtures of any of the foregoing.

The term “orifice” as used herein comprises means and methods suitable for releasing the drug from the dosage form. The expression includes one or more apertures or orifices that have been bored through the selectively permeable membrane by mechanical procedures. Alternatively, an orifice may be formed by incorporating an erodible element, such as a gelatin plug, in the selectively permeable membrane. In such cases, the pores of the selectively permeable membrane form a “passageway” for the passage of the drug. Such “passageway” formulations are described, for example, in U.S. Pat. Nos. 3,845,770 and 3,916,899, the relevant disclosures of which are incorporated herein by reference for this purpose.

The osmotic pumps useful in accordance with this invention may be manufactured by techniques known in the art. For example, the drug and other ingredients may be milled together and pressed into a solid having the desired dimensions (e.g., corresponding to the first compartment). The swellable polymer is then formed, placed in contact with the drug, and both are surrounded with the selectively permeable agent. If desired, the drug component and polymer component may be pressed together before applying the selectively permeable membrane. The selectively permeable membrane may be applied by any suitable method, for example, by molding, spraying, or dipping.

Membrane-Controlled Dosage Forms

The modified release formulations of the present invention may also be provided as membrane controlled formulations. Membrane controlled formulations of the present invention can be made by preparing a rapid release core, which may be a monolithic (e.g., tablet) or multi-unit (e.g., pellet) type, and coating the core with a membrane. The membrane-controlled core can then be further coated with a functional coating. In between the membrane-controlled core and functional coating, a barrier or sealant may be applied. Details of membrane-controlled dosage forms are provided below.

In one embodiment, 2-(3,4-dimethoxyphenol)-2-isopropyl-6-azaheptanitril, a derivative thereof or a pharmaceutically acceptable salt thereof may be provided in a multiparticulate membrane controlled formulation. 2-(3,4-dimethoxyphenol)-2-isopropyl-6-azaheptanitril, a derivative thereof or a pharmaceutically acceptable salt thereof may be formed into an active core by applying the drug to a nonpareil seed having an average diameter in the range of about 0.4 to about 1.1 mm or about 0.85 to about 1.00 mm. 2-(3,4-dimethoxyphenol)-2-isopropyl-6-azaheptanitril, a derivative thereof or a pharmaceutically acceptable salt thereof may be applied with or without additional excipients onto the inert cores, and may be sprayed from solution or suspension using a fluidized bed coater (e.g., Wurster coating) or pan coating system. Alternatively, the 2-(3,4-dimethoxyphenol)-2-isopropyl-6-azaheptanitril, a derivative thereof or a pharmaceutically acceptable salt thereof may be applied as a powder onto the inert cores using a binder to bind the 2-(3,4-dimethoxyphenol)-2-isopropyl-6-azaheptanitril onto the cores. Active cores may also be formed by extrusion of the core with suitable plasticizers (described below) and any other processing aids as necessary.

The modified release formulations of the present invention comprise at least one polymeric material, which may be applied as a membrane coating to the drug-containing cores. Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(ethylene oxide), poly(ethylene terephthalate), poly(vinyl isobutyl ether), poly(vinyl acetate), poly(vinyl chloride) or polyurethane, and/or mixtures thereof.

EUDRAGIT™ polymers (available from Rohm Pharma) are polymeric lacquer substances based on acrylates and/or methacrylates. A suitable polymer that is freely permeable to the active ingredient and water is EUDRAGIT™ RL. A suitable polymer that is slightly permeable to the active ingredient and water is EUDRAGIT™ RS. Other suitable polymers which are slightly permeable to the active ingredient and water, and exhibit a pH-dependent permeability include, but are not limited to, EUDRAGIT™ L, EUDRAGIT™ S, and EUDRAGIT™ E.

EUDRAGIT™ RL and RS are acrylic resins comprising copolymers of acrylic and methacrylic acid esters with a low content of quaternary ammonium groups. The ammonium groups are present as salts and give rise to the permeability of the lacquer films. EUDRAGIT™ RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT™ L is an anionic polymer synthesized from methacrylic acid and methacrylic acid methyl ester. It is insoluble in acids and pure water. It becomes soluble in neutral to weakly alkaline conditions. The permeability of EUDRAGIT™ L is pH dependent. Above pH 5.0, the polymer becomes increasingly permeable.

In one embodiment comprising a membrane-controlled dosage form, the polymeric material comprises methacrylic acid co-polymers, ammonio methacrylate co-polymers, or a mixture thereof. Methacrylic acid co-polymers such as EUDRAGIT™ S and EUDRAGIT™ L (Rohm Pharma) are particularly suitable for use in the controlled release formulations of the present invention. These polymers are gastroresistant and enterosoluble polymers. Their polymer films are insoluble in pure water and diluted acids. They dissolve at higher pHs, depending on their content of carboxylic acid. EUDRAGIT™ S and EUDRAGIT™ L can be used as single components in the polymer coating or in combination in any ratio. By using a combination of the polymers, the polymeric material may exhibit a solubility at a pH between the pHs at which EUDRAGIT™ L and EUDRAGIT™ S are separately soluble.

The membrane coating may comprise a polymeric material comprising a major proportion (i.e., greater than 50% of the total polymeric content) of one or more pharmaceutically acceptable water-soluble polymers, and optionally a minor proportion (i.e., less than 50% of the total polymeric content) of one or more pharmaceutically acceptable water-insoluble polymers. Alternatively, the membrane coating may comprise a polymeric material comprising a major proportion (i.e., greater than 50% of the total polymeric content) of one or more pharmaceutically acceptable water-insoluble polymers, and optionally a minor proportion (i.e., less than 50% of the total polymeric content) of one or more pharmaceutically acceptable water-soluble polymers.

Ammonio methacrylate co-polymers such as Eudragit RS and Eudragit RL (Rohm Pharma) are suitable for use in the controlled release formulations of the present invention. These polymers are insoluble in pure water, dilute acids, buffer solutions, or digestive fluids over the entire physiological pH range. The polymers swell in water and digestive fluids independently of pH. In the swollen state they are then permeable to water and dissolved actives. The permeability of the polymers depends on the ratio of ethylacrylate (EA), methyl methacrylate (MMA), and trimethylammonioethyl methacrylate chloride (TAMCI) groups in the polymer. Those polymers having EA:MMA:TAMCI ratios of 1:2:0.2 (Eudragit RL) are more permeable than those with ratios of 1:2:0.1 (Eudragit RS). Polymers of Eudragit RL are insoluble polymers of high permeability. Polymers of Eudragit RS are insoluble films of low permeability.

The ammonio methacrylate co-polymers may be combined in any desired ratio. For example, a ratio of Eudragit RS:Eudragit RL (90:10) may be used. The ratios may furthermore be adjusted to provide a delay in release of the drug. For example, the ratio of Eudragit RS:Eudragit RL may be about 100:0 to about 80:20, about 100:0 to about 90:10, or any ratio in between. In such formulations, the less permeable polymer Eudragit RS would generally comprise the majority of the polymeric material.

The ammonio methacrylate co-polymers may be combined with the methacrylic acid co-polymers within the polymeric material in order to achieve the desired delay in release of the drug. Ratios of ammonio methacrylate co-polymer (e.g., Eudragit RS) to methacrylic acid co-polymer in the range of about 99:1 to about 20:80 may be used. The two types of polymers can also be combined into the same polymeric material, or provided as separate coats that are applied to the core.

In addition to the Eudragit polymers described above, a number of other such copolymers may be used to control drug release. These include methacrylate ester co-polymers (e.g., Eudragit NE 30D). Further information on the Eudragit polymers can be found in “Chemistry and Application Properties of Polymethacrylate Coating Systems,” in Aqueous Polymeric Coatings for Pharmaceutical Dosage Forms, ed. James McGinity, Marcel Dekker Inc., New York, pg 109-114.

The coating membrane may further comprise at least one soluble excipient so as to increase the permeability of the polymeric material. Suitably, the soluble excipient is selected from among a soluble polymer, a surfactant, an alkali metal salt, an organic acid, a sugar, and a sugar alcohol. Such soluble excipients include, but are not limited to, polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid, sugars such as dextrose, fructose, glucose, lactose and sucrose, sugar alcohols such as lactitol, maltitol, mannitol, sorbitol and xylitol, xanthan gum, dextrins, and maltodextrins. In some embodiments, polyvinyl pyrrolidone, mannitol, and/or polyethylene glycol can be used as soluble excipients. The soluble excipient(s) may be used in an amount of from about 1% to about 10% by weight, based on the total dry weight of the polymer.

In another embodiment, the polymeric material comprises at least one water-insoluble polymer, which are also insoluble in gastrointestinal fluids, and at least one water-soluble pore-forming compound. For example, the water-insoluble polymer may comprise a terpolymer of polyvinylchloride, polyvinylacetate, and/or polyvinylalcohol. Suitable water-soluble pore-forming compounds include, but are not limited to, saccharose, sodium chloride, potassium chloride, polyvinylpyrrolidone, and/or polyethyleneglycol. The pore-forming compounds may be uniformly or randomly distributed throughout the water-insoluble polymer. Typically, the pore-forming compounds comprise about 1 part to about 35 parts for each about 1 to about 10 parts of the water-insoluble polymers.

When such dosage forms come in to contact with the dissolution media (e.g., intestinal fluids), the pore-forming compounds within the polymeric material dissolve to produce a porous structure through which the drug diffuses. Such formulations are described in more detail in U.S. Pat. No. 4,557,925, which relevant part is incorporated herein by reference for this purpose. The porous membrane may also be coated with an enteric coating, as described herein, to inhibit release in the stomach.

In one embodiment, such pore forming controlled release dosage forms comprise 2-(3,4-dimethoxyphenol)-2-isopropyl-6-azaheptanitril, a derivative thereof or a pharmaceutically acceptable salt thereof; a filler, such as starch, lactose, or microcrystalline cellulose (AVICEL™); a binder/controlled release polymer, such as hydroxypropyl methylcellulose or polyvinyl pyrrolidone; a disintegrant, such as, EXPLOTAB™, crospovidone, or starch; a lubricant, such as magnesium stearate or stearic acid; a surfactant, such as sodium lauryl sulphate or polysorbates; and a glidant, such as colloidal silicon dioxide (AEROSIL™) or talc.

The polymeric material may also include one or more auxiliary agents such as fillers, plasticizers, and/or anti-foaming agents. Representative fillers include talc, fumed silica, glyceryl monostearate, magnesium stearate, calcium stearate, kaolin, colloidal silica, gypsum, micronized silica, and magnesium trisilicate. The quantity of filler used typically ranges from about 2% to about 300% by weight, and can range from about 20 to about 100%, based on the total dry weight of the polymer. In one embodiment, talc is the filler.

The coating membranes, and functional coatings as well, can also include a material that improves the processing of the polymers. Such materials are generally referred to as plasticizers and include, for example, adipates, azelates, benzoates, citrates, isoebucates, phthalates, sebacates, stearates, and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 10% to about 50%, for example, about 10%, 20%, 30%, 40%, or 50%, based on the weight of the dry polymer.

Anti-foaming agents can also be included. In one embodiment, the anti-foaming agent is simethicone. The amount of anti-foaming agent used typically comprises from about 0% to about 0.5% of the final formulation.

The amount of polymer to be used in the membrane controlled formulations is typically adjusted to achieve the desired drug delivery properties, including the amount of drug to be delivered, the rate and location of drug delivery, the time delay of drug release, and the size of the multiparticulates in the formulation. The amount of polymer applied typically provides an about 10% to about 100% weight gain to the cores. In one embodiment, the weight gain from the polymeric material ranges from about 25% to about 70%.

The combination of all solid components of the polymeric material, including co-polymers, fillers, plasticizers, and optional excipients and processing aids, typically provides an about 10% to about 450% weight gain on the cores. In one embodiment, the weight gain is about 30% to about 160%.

The polymeric material can be applied by any known method, for example, by spraying using a fluidized bed coater (e.g., Wurster coating) or pan coating system. Coated cores are typically dried or cured after application of the polymeric material. Curing means that the multiparticulates are held at a controlled temperature for a time sufficient to provide stable release rates. Curing can be performed, for example, in an oven or in a fluid bed drier. Curing can be carried out at any temperature above room temperature.

A sealant or barrier can also be applied to the polymeric coating. A sealant or barrier layer may also be applied to the core prior to applying the polymeric material. A sealant or barrier layer is not intended to modify the release of 2-(3,4-dimethoxyphenol)-2-isopropyl-6-azaheptanitril, a derivative thereof or a pharmaceutically acceptable salt. Suitable sealants or barriers are permeable or soluble agents such as hydroxypropyl methylcellulose, hydroxypropyl cellulose, hydroxypropyl ethylcellulose, and xanthan gum.

Other agents can be added to improve the processability of the sealant or barrier layer. Such agents include talc, colloidal silica, polyvinyl alcohol, titanium dioxide, micronized silica, fumed silica, glycerol monostearate, magnesium trisilicate and magnesium stearate, or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY WHITE Y-1-7000 and OPADRY OY/B/28920 WHITE, each of which is available from Colorcon Limited, England.

The invention also provides an oral dosage form containing a multiparticulate 2-(3,4-dimethoxyphenol)-2-isopropyl-6-azaheptanitril, a derivative thereof or a pharmaceutically acceptable salt thereof, formulation as hereinabove defined, in the form of caplets, capsules, particles for suspension prior to dosing, sachets, or tablets. When the dosage form is in the form of tablets, the tablets may be disintegrating tablets, fast dissolving tablets, effervescent tablets, fast melt tablets, and/or mini-tablets. The dosage form can be of any shape suitable for oral administration of a drug, such as spheroidal, cube-shaped oval, or ellipsoidal. The dosage forms can be prepared from the multiparticulates in a manner known in the art and include additional pharmaceutically acceptable excipients, as desired.

All of the particular embodiments described above, including but not limited to, matrix-based, osmotic pump-based, soft gelatin capsules, and/or membrane-controlled forms, which may further take the form of monolithic and/or multi-unit dosage forms, may have a functional coating. Such coatings generally serve the purpose of delaying the release of the drug for a predetermined period. For example, such coatings may allow the dosage form to pass through the stomach without being subjected to stomach acid or digestive juices. Thus, such coatings may dissolve or erode upon reaching a desired point in the gastrointestinal tract, such as the upper intestine.

Such functional coatings may exhibit pH-dependent or pH-independent solubility profiles. Those with pH-independent profiles generally erode or dissolve away after a predetermined period, and the period is generally directly proportional to the thickness of the coating. Those with pH-dependent profiles, on the other hand, may maintain their integrity while in the acid pH of the stomach, but quickly erode or dissolve upon entering the more basic upper intestine.

Thus, a matrix-based, osmotic pump-based, or membrane-controlled formulation may be further coated with a functional coating that delays the release of the drug. For example, a membrane-controlled formulation may be coated with an enteric coating that delays the exposure of the membrane-controlled formulation until the upper intestine is reached. Upon leaving the acidic stomach and entering the more basic intestine, the enteric coating dissolves. The membrane-controlled formulation then is exposed to gastrointestinal fluid, and then releases 2-(3,4-dimethoxyphenol)-2-isopropyl-6-azaheptanitril, a derivative thereof or a pharmaceutically acceptable salt thereof over an extended period, in accordance with the invention. Examples of functional coatings such as these are well known to those in the art.

Any of the oral dosage forms described herein may be provided in the form of caplets, capsules, beads, granules, particles for suspension prior to dosing, sachets, or tablets. When the dosage form is in the form of tablets, the tablets may be disintegrating tablets, fast dissolving tablets, effervescent tablets, fast melt tablets, and/or mini-tablets. The dosage form can be of any shape suitable for oral administration of a drug, such as spheroidal, cube-shaped oval, or ellipsoidal.

The thickness of the polymer in the formulations, the amounts and types of polymers, and the ratio of water-soluble polymers to water-insoluble polymers in the modified-release formulations are generally selected to achieve a desired release profile of 2-(3,4-dimethoxyphenol)-2-isopropyl-6-azaheptanitril, a derivative thereof or a pharmaceutically acceptable salt thereof. For example, by increasing the amount of water-insoluble-polymer relative to the water-soluble polymer, the release of the drug may be delayed or slowed.

The amount of the dose administered, as well as the dose frequency, will vary depending on the particular dosage form used and route of administration. The amount and frequency of administration will also vary according to the age, body weight, and response of the individual subject. Typical dosing regimens can readily be determined by a competent physician without undue experimentation. It is also noted that the clinician or treating physician will know how and when to interrupt, adjust, or terminate therapy in conjunction with individual subject response.

In general, the total daily dosage for treating, preventing, and/or managing the conditions with any of the formulations according to the present invention is from about 1 mg to about 1000 mg, or about 1, 5, 10, 15, 20, 25, 30, 35, 40, 45, 50, 55, 60, 70, 80, 90, 100, 120, 140, 150, 160, 180, 200, 250, 300, 350, 400, 450, 500, 550, 600, 650, 700, 750, 800, 850, 900, 950, or 1000 mg, or any number in between, of 2-(3,4-dimethoxyphenol)-2-isopropyl-6-azaheptanitril, a derivative thereof, or a pharmaceutically acceptable salt thereof. For example, for an orally administered dosage form, the total daily dose may range from about 30 mg to about 800 mg, or from about 60 mg to about 600 mg, or from about 120 mg to about 400 mg, or from about 120 mg to about 200 mg. Accordingly, a single oral dose may be formulated to contain about 1, 5, 10, 15, 20, 25, 30, 35, 40, 45, 50, 55, 60, 70, 80, 90, 100, 120, 140, 150, 160, 180, 200, 220, 240, 250, 260, 280, 300, 320, 340, 350, 360, 380, 400, 420, 440, 450, 460, 480, 500, 520, 540, 550, 560, 580, or 600 mg, or any number in between, of 2-(3,4-dimethoxyphenol)-2-isopropyl-6-azaheptanitril, a derivative thereof or a pharmaceutically acceptable salt thereof. The pharmaceutical compositions containing 2-(3,4-dimethoxyphenol)-2-isopropyl-6-azaheptanitril, a derivative thereof or a pharmaceutically acceptable salt thereof may be administered in single or divided doses 1, 2, 3, 4, or more times each day. Alternatively, the dose may be delivered once every 2, 3, 4, 5, or more days. In one embodiment, the pharmaceutical compositions are administered once per day.

Any of the pharmaceutical compositions and dosage forms described herein may further comprise at least one additional pharmaceutically active compound other than 2-(3,4-dimethoxyphenol)-2-isopropyl-6-azaheptanitril, a derivative thereof or a pharmaceutically acceptable salt thereof that may or may not have serotonin transporter activity. Such compounds may be included to treat, prevent, and/or manage the same condition being treated, prevented, and/or managed with 2-(3,4-dimethoxyphenol)-2-isopropyl-6-azaheptanitril, a derivative thereof or a pharmaceutically acceptable salt thereof, or a different one. Those of skill in the art are familiar with examples of the techniques for incorporating additional active ingredients into compositions comprising 2-(3,4-dimethoxyphenol)-2-isopropyl-6-azaheptanitril, a derivative thereof or a pharmaceutically acceptable salt thereof. Alternatively, such additional pharmaceutical compounds may be provided in a separate formulation and co-administered to a subject with 2-(3,4-dimethoxyphenol)-2-isopropyl-6-azaheptanitril, a derivative thereof or a pharmaceutically acceptable salt thereof composition according to the present invention. Such separate formulations may be administered before, after, or simultaneously with the administration of 2-(3,4-dimethoxyphenol)-2-isopropyl-6-azaheptanitril, a derivative thereof or a pharmaceutically acceptable salt thereof compositions of the present invention.

The invention is further illustrated by reference to the following examples. It will be apparent to those skilled in the art that many modifications, both to the materials and methods, may be practiced without departing from the purpose and scope of the invention.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

### In vitro Binding Assay of (R)-2-(3,4-dimethoxyphenol)-2-isopropyl-6-azaheptanitril on the Serotonin Transporter

Basically, the assay that was performed was an antagonist radioligand study using human recombinant CHO cells, following to the general method described in Tatsumi et al., Eur. J. Pharmacol. 368: 277-283 (1999). For its discussion of the assay method, Tatsumi et al. is incorporated herein by reference in its entirety.

Experimental Conditions

| | | | | | Method of |
|---|---|---|---|---|---|
| Assay | Ligand | Conc. | Non Specific | Incubation | Detection |
| 5-HT transporter (h) | [³H]imipramine | 2 nM | imipramine | 60 | Scintillation |
| (antagonist radioligand) | | | (10 μM) | min./22° C. | counting |

Analysis and Expression of Results

The specific ligand binding to the receptors is defined as the difference between the total binding and the nonspecific binding determined in the presence of an excess of unlabelled ligand.

The results are expressed as a percent of control specific binding ((measured specific binding/control specific binding)×100) and as a percent inhibition of control specific binding (100−((measured specific binding/control specific binding)×100)) obtained in the presence of (R)-2-(3,4-dimethoxyphenol)-2-isopropyl-6-azaheptanitril.

The IC₅₀ values (concentration causing a half-maximal inhibition of control specific binding) and Hill coefficients (nH) were determined by non-linear regression analysis of the competition curves generated with mean replicate values using Hill equation curve fitting (Y=D+[(A−D)/(1+(C/C₅₀)^{nH})], where Y=specific binding, D=minimum specific binding, A=maximum specific binding, C=compound concentration, C₅₀=IC₅₀, and nH=slope factor). This analysis was performed using a software developed at Cerep (Hill software) and validated by comparison with data generated by the commercial software SigmaPlot® 4.0 for Windows® (© 1997 by SPSS Inc.).

The inhibition constants (Kᵢ) were calculated using the Cheng Prusoff equation (Kᵢ=IC₅₀/(1+(L/K_{D})), where L=concentration of radioligand in the assay, and K_{D}=affinity of the radioligand for the receptor).

In vitro Uptake Assay

Briefly, the uptake assay was performed using rat brain synaptosomes according to the method generally described in Perovic and Muller, Arzneim-Forsch. Drug Res., 45: 1145-1148 (1995). For its disclosure of the above-noted assay, Perovic and Muller is incorporated herein by reference in its entirety.

Experimental Conditions

| | Substrate/Stimulus/ | | | Method of |
|---|---|---|---|---|
| Assay | Tracer | Incubation | Reaction Product | Detection |
| 5-HT | [³H]5-HT | 15 min./37° C. | [³H]5-HT incorporation | Scintillation |
| uptake | (0.2 μCi/ml) | | into synaptosomes | counting |

Analysis and Expression of Results

The results are expressed as a percent of control specific activity ((measured specific activity/control specific activity)×100) obtained in the presence of (R)-2-(3,4-dimethoxyphenol)-2-isopropyl-6-azaheptanitril.

The IC₅₀ values (concentration causing a half-maximal inhibition of control specific activity) and Hill coefficients (nH) were determined by non-linear regression analysis of the inhibition curves generated with mean replicate values using Hill equation curve fitting (Y=D+[(A−D)/(1+(C/C₅₀)^{nH})], where Y=specific activity, D=minimum specific activity, A=maximum specific activity, C=compound concentration, C₅₀=IC₅₀ and nH=slope factor). This analysis was performed using a software developed at Cerep (Hill software) and validated by comparison with data generated by the commercial software SigmaPlot® 4.0 for Windows® (© 1997 by SPSS Inc.).

Reference Compounds

In each experiment, the respective reference compound was tested concurrently with (R)-2-(3,4-dimethoxyphenol)-2-isopropyl-6-azaheptanitril.

Results

In vitro Binding Assays

The mean values for the effects of (R)-2-(3,4-dimethoxyphenol)-2-isopropyl-6-azaheptanitril are summarized in Table 1-1. The individual data obtained with (R)-2-(3,4-dimethoxyphenol)-2-isopropyl-6-azaheptanitril are reported in Table 1-2. The IC₅₀ and Kᵢ values for each reference compound are indicated in Table 1-3. The IC₅₀ and Kᵢ values determined for (R)-2-(3,4-dimethoxyphenol)-2-isopropyl-6-azaheptanitril are indicated in Table 1-4.

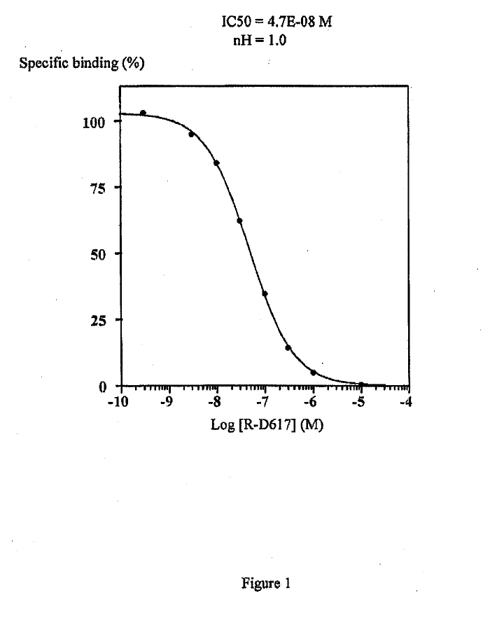

The corresponding competition curve obtained with (R)-2-(3,4-dimethoxyphenol)-2-isopropyl-6-azaheptanitril is shown in FIG. 1. The individual data obtained with (R)-2-(3,4-dimethoxyphenol)-2-isopropyl-6-azaheptanitril are reported in Table 1-5. The IC₅₀ and Kᵢ values for the reference compound are indicated in Table 1-6.

TABLE 1-1
Summary Results

| | | Test | % Inhibition |
|---|---|---|---|
| | | Concen- | of Control |
| Assay | Compound | tration (M) | Specific Binding |
| 5-HT | (R)-2-(3,4- | 1.0E−05 | 100 |
| transporter | dimethoxyphenol)- | | |
| (h)(antagonist | 2-isopropyl-6- | | |
| radioligand) | azaheptanitril | | |

TABLE 1-2
Individual Data

| | | Test | % of Control | | |
|---|---|---|---|---|---|
| | | Concen- | Specific Binding | | |
| Assay | Compound | tration (M) | 1^{st} | 2^{nd} | Mean |
| 5-HT | (R)-2-(3,4- | 1.0E−05 | −0.6 | 0.8 | 0.1 |
| transporter | dimethoxyphenol)- | | | | |
| (h)(antagonist | 2-isopropyl-6- | | | | |
| radioligand) | azaheptanitril | | | | |

TABLE 1-3
Reference Compound Data

| Assay | IC₅₀ (M) | Kᵢ (M) | n_{H} |
|---|---|---|---|
| 5-HT transporter (h) | 5.4E−09 | 2.5E−09 | 0.9 |
| (antagonist radioligand) | | | |
| Reference compound: imipramine | | | |

TABLE 1-4
IC₅₀ Determination: Summary Results

| Assay | Compound | IC₅₀ (M) | Kᵢ (M) | n_{H} |
|---|---|---|---|---|
| 5-HT | (R)-2-(3,4- | 4.7E−08 | 2.2E−08 | 1.0 |
| transporter | dimethoxyphenol)- | | | |
| (h)(antagonist | 2-isopropyl-6- | | | |
| radioligand) | azaheptanitril | | | |

TABLE 1-5
IC₅₀ Determination: Individual Data

| | | Test | % of Control | | |
|---|---|---|---|---|---|
| | | Concen- | Specific Binding | | |
| Assay | Compound | tration (M) | 1^{st} | 2^{nd} | Mean |
| 5-HT | (R)-2-(3,4- | 3.0E−10 | 104.1 | 102.0 | 103.1 |
| transporter | dimethoxyphenol)- | 3.0E−09 | 101.5 | 88.4 | 94.9 |
| (h) (antagonist | 2-isopropyl-6- | 1.0E−08 | 83.3 | 84.8 | 84.0 |
| radioligand) | azaheptanitril | 3.0E−08 | 66.6 | 58.0 | 62.3 |
| | | 1.0E−07 | 36.5 | 32.5 | 34.5 |
| | | 3.0E−07 | 15.3 | 13.1 | 14.2 |
| | | 1.0E−06 | 5.9 | 3.7 | 4.8 |
| | | 1.0E−05 | 0.5 | 0.2 | 0.3 |

TABLE 1-6
Reference Compound Data

| Assay | IC₅₀ (M) | Kᵢ (M) | n_{H} |
|---|---|---|---|
| 5-HT transporter (h) | 8.5E−09 | 3.9E−09 | 1.2 |
| (antagonist radioligand) | | | |
| Reference compound: imipramine | | | |

The corresponding competition curve obtained with (R)-2-(3,4-dimethoxyphenol)-2-isopropyl-6-azaheptanitril is shown in FIG. 1. The individual data obtained with (R)-2-(3,4-dimethoxyphenol)-2-isopropyl-6-azaheptanitril are reported in Table 1-5. The IC₅₀ and Kᵢ values for the reference compound are indicated in Table 1-6.

In vitro Uptake Assay

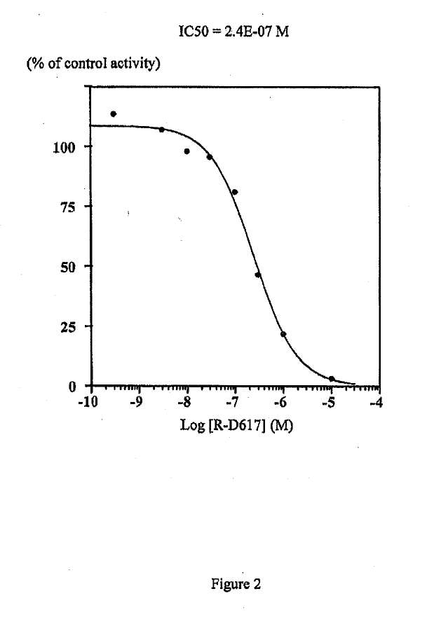

The IC₅₀ value determined for (R)-2-(3,4-dimethoxyphenol)-2-isopropyl-6-azaheptanitril is indicated in Table 2-1. The corresponding inhibition curve obtained with (R)-2-(3,4-dimethoxyphenol)-2-isopropyl-6-azaheptanitril is shown in FIG. 2. The individual data obtained with (R)-2-(3,4-dimethoxyphenol)-2-isopropyl-6-azaheptanitril are reported in Table 2-2. The IC₅₀ value for the reference compound is indicated in Table 2-3.

TABLE 2-1
IC₅₀ Determination: Summary Results

| Assay | Compound | IC₅₀ (M) |
|---|---|---|
| 5-HT uptake | (R)-2-(3,4-dimethoxyphenol)-2- | 2.4E−07 |
| | isopropyl-6-azaheptanitril | |

TABLE 2-2
IC₅₀ Determination: Individual Data

| | | Test | | | |
|---|---|---|---|---|---|
| | | Concentration | % of Control Values | | |
| Assay | Compound | (M) | 1^{st} | 2^{nd} | Mean |
| 5-HT uptake | (R)-2-(3,4- | 3.0E−10 | 113.5 | 113.3 | 113.4 |
| | dimethoxyphenol)- | 3.0E−09 | 100.2 | 113.4 | 106.8 |
| | 2-isopropyl-6- | 1.0E−08 | 101.7 | 94.0 | 97.9 |
| | azaheptanitril | 3.0E−08 | 108.1 | 82.8 | 95.4 |
| | | 1.0E−07 | 79.8 | 81.9 | 80.8 |
| | | 3.0E−07 | 47.8 | 44.9 | 46.3 |
| | | 1.0E−06 | 22.1 | 21.1 | 21.6 |
| | | 1.0E−05 | 1.0 | 5.2 | 3.1 |

TABLE 2-3
Reference Compound Data

| Assay | IC₅₀ (M) |
|---|---|
| 5-HT uptake | 4.1E−08 |
| Reference compound: imipramine | |

As can be seen from the results obtained in this Example, (R)-2-(3,4-dimethoxyphenol)-2-isopropyl-6-azaheptanitril was surprisingly discovered to have strong activity on the serotonin transporter. It is therefore believed that (R)-2-(3,4-dimethoxyphenol)-2-isopropyl-6-azaheptanitril will have utilities in the treatments of diseases and conditions that involve the serotonin transporter.

Inhibition of PAH Development

Experimental pulmonary arterial hypertension (PAH) was induced in male wistar rats (Charles River Laboratories) weighing between 250 and 300 g by subcutaneous injection of 60 mg/kg Monocrotaline (Met) on Day 1. The Mct (Sigma Aldrich) was dissolved in 1N HCl at a concentration of 300 mg/ml as per M. Rey et al., Current Protocols in Pharmacology, 5.56.1-5.56.11, September 2009, which document is incorporated by reference herein in its entirety. Mct was then neutralized by adding 1N NaOH and diluted with sterile distilled water to a final concentration of 20 mg/ml. An injection volume of 3 ml/kg was used to achieve the 60 mg/kg dose.

A total of 44 rats were injected as follows: 15 rats were injected subcutaneously with Mct (untreated PAH controls), 15 rats were injected subcutaneously with Mct followed by daily IP injection with 5 mg/kg (R)-2-(3,4-dimethoxyphenol)-2-isopropyl-6-azaheptanitril for 21 days (R-D617 treated group), and 14 rats were injected subcutaneously with neutralized solvent having no Mct (non-PAH controls (sham)).

All animals were then housed (allowed food and water ad lib). The animals were anaesthetized 21 days after injection, and pulmonary arterial pressure was measured and expressed as mean pulmonary arterial pressure (mPAP mmHg). A terminal sample was also obtained and used to obtain terminal drug concentrations. Following exsanguination, cardiac tissues were excised and used to assess the wet weight of the right cardiac ventricle. Concentrations of R-verapamil and (R)-2-(3,4-dimethoxyphenol)-2-isopropyl-6-azaheptanitril in the terminal samples were measured by HPLC with a lower limit of quantification of 1 ng/ml and 10 ng/ml, respectively.

Results

One animal died in each of the 3 groups during the 21-day period and therefore results are presented for the 14, 14, and 13 surviving animals in the R-D617 treated, untreated PAH control, and sham groups, respectively:

TABLE 3
Summary of R-D617 Treatment on PAH Development

| | mPAP (mmHg) | Right Ventricle Weight |
|---|---|---|
| Group | (Mean +/− SEM) | (Mean +/− SEM) |
| R-D617 treated | 30.0 +/− 2.5 | 288.7 +/− 13.4 |
| Untreated PAH control | 36.0 +/− 1.5 | 317.9 +/− 13.9 |
| Sham | 14.7 +/− 0.9 | 167.4 +/− 7.5 |

Furthermore, the mean terminal plasma concentration of (R)-2-(3,4-dimethoxyphenol)-2-isopropyl-6-azaheptanitril was determined to be 18.5 ng/ml for the R-D617 treated group, whereas there was no quantifiable R-verapamil measured in any of the animals in this group.

As disclosed above, Mct induced PAH as evidenced by the increased mPAP compared with the sham (Mct-negative) control group. In addition there was a matching increase in right ventricle weight in the untreated PAH control group, which increase is an index of right-sided hypertrophy, which is characteristic of PAH. Treatment with (R)-2-(3,4-dimethoxyphenol)-2-isopropyl-6-azaheptanitril was shown to attenuate the increase in mPAP, as well as the increase in right ventricle weight. Furthermore, terminal plasma concentrations confirm that R-D617 treatment results in (R)-2-(3,4-dimethoxyphenol)-2-isopropyl-6-azaheptanitril exposure, but is free of any quantifiable (<1 ng/ml) R-verapamil. Thus, (R)-2-(3,4-dimethoxyphenol)-2-isopropyl-6-azaheptanitril can prevent or inhibit PAH and/or PAH development.

Given the selective and potent activity of (R)-2-(3,4-dimethoxyphenol)-2-isopropyl-6-azaheptanitril to inhibit the serotonin transporter, the therapeutic applications of this compound are based on those conditions where a reduction in cell or tissue uptake of serotonin (5-HT) is beneficial. These indications include uses where inhibition of the serotonin transporter is already established with approved therapies, e.g., depression and other affective disorders and PMS, but also where inhibition of the serotonin transporter has been shown experimentally or speculated as being beneficial.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of treating pulmonary arterial hypertension comprising administering a composition comprising a therapeutically effective amount of (R)-2-(3,4-dimethoxyphenol)-2-isopropyl-6-azaheptanitril or a pharmaceutically acceptable salt thereof to a patient in need thereof, wherein such administration results in a therapeutically effective plasma level of (R)-2-(3,4-dimethoxyphenol)-2-isopropyl-6-azaheptanitril, in the absence of a therapeutically effective level of R-verapamil.

2. The method according to claim 1, wherein the composition further comprises at least one pharmaceutically acceptable excipient.

3. The method according to claim 1, wherein the (R)-2-(3,4-dimethoxyphenol)-2-isopropyl-6-azaheptanitril or a pharmaceutically acceptable salt thereof, is present in the composition in an amount ranging from about 1 mg to about 1000 mg.

4. The method according to claim 3, wherein the (R)-2-(3,4-dimethoxyphenol)-2-isopropyl-6-azaheptanitril or a pharmaceutically acceptable salt thereof, is administered orally and in an amount ranging from about 1 mg to 800 mg per day.

5. The method according to claim 4, wherein the (R)-2-(3,4-dimethoxyphenol)-2-isopropyl-6-azaheptanitril or a pharmaceutically acceptable salt thereof, is administered orally and in an amount from about 1 mg to about 250 mg per day.

6. The method according to claim 1, wherein the unbound plasma concentration range provided by the (R)-2-(3,4-dimethoxyphenol)-2-isopropyl-6-azaheptanitril or a pharmaceutically acceptable salt thereof, is from about 1×10⁻⁵M to about 1×10⁻¹⁰M.

7. The method according to claim 1, wherein the composition comprises a therapeutically effective amount of (R)-2-(3,4-dimethoxyphenol)-2-isopropyl-6-azaheptanitril.

8. A method of reducing pulmonary arterial pressure comprising administering a composition comprising a therapeutically effective amount of (R)-2-(3,4-dimethoxyphenol)-2-isopropyl-6-azaheptanitril or a pharmaceutically acceptable salt thereof to a patient in need thereof, wherein such administration results in a therapeutically effective plasma level of (R)-2-(3,4-dimethoxyphenol)-2-isopropyl-6-azaheptanitril, in the absence of a therapeutically effective level of R-verapamil.

9. The method according to claim 8, wherein the (R)-2-(3,4-dimethoxyphenol)-2-isopropyl-6-azaheptanitril or a pharmaceutically acceptable salt thereof, is present in the composition in an amount ranging from about 1 mg to about 1000 mg.

10. The method according to claim 9, wherein the (R)-2-(3,4-dimethoxyphenol)-2-isopropyl-6-azaheptanitril or a pharmaceutically acceptable salt thereof, is administered orally and in an amount ranging from about 1 mg to 800 mg per day.

11. The method according to claim 10, wherein the (R)-2-(3,4-dimethoxyphenol)-2-isopropyl-6-azaheptanitril or a pharmaceutically acceptable salt thereof, is administered orally and in an amount from about 1 mg to about 250 mg per day.

12. The method according to claim 8, wherein the unbound plasma concentration range provided by the (R)-2-(3,4-dimethoxyphenol)-2-isopropyl-6-azaheptanitril or a pharmaceutically acceptable salt thereof, is from about 1×10⁻⁵M to about 1×10⁻¹⁰M.

13. The method according to claim 8, wherein the composition comprises a therapeutically effective amount of (R)-2-(3,4-dimethoxyphenol)-2-isopropyl-6-azaheptanitril.

14. A method of inhibiting cardiac hypertrophy comprising administering a composition comprising a therapeutically effective amount of (R)-2-(3,4-dimethoxyphenol)-2-isopropyl-6-azaheptanitril or a pharmaceutically acceptable salt thereof to a patient in need thereof, wherein such administration results in a therapeutically effective plasma level of (R)-2-(3,4-dimethoxyphenol)-2-isopropyl-6-azaheptanitril, in the absence of a therapeutically effective level of R-verapamil.

15. The method according to claim 14, wherein the (R)-2-(3,4-dimethoxyphenol)-2-isopropyl-6-azaheptanitril or a pharmaceutically acceptable salt thereof, is present in the composition in an amount ranging from about 1 mg to about 1000 mg.

16. The method according to claim 15, wherein the (R)-2-(3,4-dimethoxyphenol)-2-isopropyl-6-azaheptanitril or a pharmaceutically acceptable salt thereof, is administered orally and in an amount ranging from about 1 mg to 800 mg per day.

17. The method according to claim 16, wherein the (R)-2-(3,4-dimethoxyphenol)-2-isopropyl-6-azaheptanitril or a pharmaceutically acceptable salt thereof, is administered orally and in an amount from about 1 mg to about 250 mg per day.

18. The method according to claim 14, wherein the unbound plasma concentration range provided by the (R)-2-(3,4-dimethoxyphenol)-2-isopropyl-6-azaheptanitril or a pharmaceutically acceptable salt thereof, is from about 1×10⁻⁵M to about 1×10⁻¹⁰M.

19. The method according to claim 14, wherein the composition comprises a therapeutically effective amount of (R)-2-(3,4-dimethoxyphenol)-2-isopropyl-6-azaheptanitril.

20. A composition comprising from about 1 mg to about 1000 mg of the (R)-isomer of 2-(3,4-dimethoxyphenol)-2-isopropyl-6-azaheptanitril and at least one pharmaceutically acceptable excipient.
